(19) United States
(12) Patent Application Publication
(10) Pub. No.: US 2008/0261209 A1
(43) Pub. Date: Oct. 23, 2008

(54) Electrophoretic Separation Method for Analyzing Gene Expression

(76) Inventors: Kai Sohn, Schwabisch-Gmund (DE); Steffen Rupp, Stuttgart (DE)

Correspondence Address: OSTROLENK FABER GERB & SOFFEN, 1180 AVENUE OF THE AMERICAS, NEW YORK, NY, 100368403, US

(21) Appl. No.: 11/664,403

(22) PCT Filed: Sep. 30, 2005
(86) PCT No.: PCT/EP2005/010566

§ 371 Date: Feb. 27, 2008

(30) Foreign Application Priority Data
Oct. 1, 2004 (DE) 10 2004 048 334.5

(51) Int. Cl. C12Q 1/68

(52) U.S. Cl. 435/6

(57) ABSTRACT

The following invention relates to an improved method of quantitative or qualitative analysis of gene expression of a biological material.

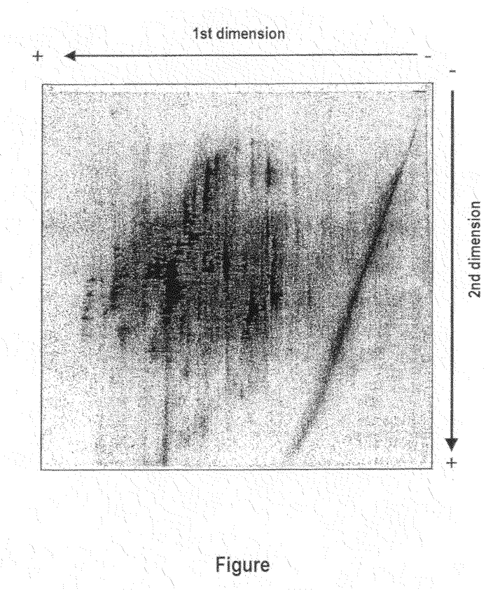

The present invention relates to an improved method of quantitative or qualitative analysis of gene expression of a biological material.

Comprehensive functional characterization of genes plays a central part nowadays in the area of biosciences and medicine but also food technology. Of great importance here are “gene expression studies”, i.e. qualitative and quantitative analyses for determining the genetic activities of an organism. Currently, gene expression analyses are carried out by means of the “microarray technology”. Methods based on said microarray technology, however, have serious disadvantages. Microarrays normally comprise up to several thousand different DNA fragments which act as specific probes. Preparing these probes is extremely complicated and therefore very expensive. Generation of said probes furthermore requires sequence information of the genes to be analyzed. Genome-wide expression studies therefore have previously been restricted to fully sequenced organisms. Comprehensive gene expression studies cannot be carried out on many economically relevant and complex organisms such as crop plants or useful animals, since complete genomic sequences of only a few selected higher model organisms such as humans or mice are currently available. A growing need for advantageous alternatives of the established microarray technology, which do not have the above-mentioned disadvantages, can therefore be recognized.

The present invention therefore is based on the technical problem of providing a method of quantitative or qualitative analysis of gene expression of a biological material, which method enables gene expression studies to be carried out as inexpensive and simple as possible, in particular also on complex biological materials whose sequence information is currently not available or incomplete.

The present invention solves the technical problem on which it is based by providing a method of quantitative and/or qualitative analysis of gene expression of a biological material, comprising isolating in a first step a) RNA from said biological material, obtaining in a second step b) at least one population of labeled double-stranded cDNA from said RNA, carrying out in a third step c) a multi-dimensional fractionation of the at least one cDNA population in a separating system, and carrying out in a fourth step d) a qualitative and/or quantitative evaluation of the multi-dimensionally fractionated spots obtained of the at least one cDNA population.

The invention also relates to a method of quantitative or qualitative analysis of gene expression of a biological material, comprising isolating in a first step a) at least two different total RNAs or at least two different mRNA populations from said biological material, obtaining in a second step b), in particular by reverse transcription, at least two different populations of double-stranded cDNA from the at least two different RNAs, in particular at least two different total RNAs or the at least two different mRNA populations, where appropriate amplifying said populations of double-stranded cDNA, wherein the at least two different cDNA populations obtained are labeled in each case differently during said step b), carrying out in a third step c) a multi-dimensional distribution of the at least two different cDNA populations in a separating system, and carrying out in a fourth step d) a qualitative and/or quantitative evaluation of the multidimensionally fractionated spots obtained of the at least two cDNA populations, wherein the at least two different cDNA populations are fractionated together in a separating system.

In the context of the present invention, RNA means both mRNA and total RNA. The starting material used in accordance with the invention is therefore both mRNA, in particular at least one mRNA population, and total RNA. The mRNA or mRNA population used is normally derived from total RNA, after carrying out an isolation step.

In the context of the present invention, the terms RNA, mRNA, DNA, cDNA always mean the corresponding DNA or RNA molecules, unless stated otherwise.

The invention therefore provides for a first step of providing RNA, i.e. either total RNA or at least one mRNA population of a biological material, from which subsequently at least one cDNA population is prepared, in particular by means of reverse transcription, and said cDNA population is labeled either during or after reverse transcription. A preferred embodiment of the invention may provide for amplifying the cDNA obtained, for example by means of PCR, after reverse transcription, i.e. after cDNA synthesis. In a preferred embodiment, primers which have no specificity for particular genes or particular nucleotide sequences, i.e. which are sequence-, gene- and/or organism-unspecific, may be used both for cDNA synthesis and for optional cDNA amplification. Said primers may be labeled, in particular fluorescently labeled.

In a preferred embodiment, the molecules of the cDNA population obtained, i.e. the cDNA fragments, are cut with the aid of DNA-cleaving enzymes, for example restriction endonucleases, preferably sequence-specific restriction endo-nucleases, thereby determining especially the average molecular weight of said cDNA population.

This is followed by fractionating in a separating system, in particular an electrophoretic separating system, preferably a gel-electrophoretic separating system, the at least one cDNA population obtained in at least two different dimensions, for example two or three dimensions, in such a way that at least one cDNA population produces a multi-dimensional pattern of spatially separated spots, which pattern may be evaluated qualitatively and/or quantitatively. The present method is advantageous in that it enables double-stranded DNA to be fractionated with high resolution, independently of the principle of the hybridization of complementary DNA to specific gene probes, which hybridization is employed in conventional microarray technologies. The present method makes it possible and serves to analyze, also identify, biological materials whose sequence information is not yet available or is currently incomplete, i.e. in particular to analyze gene expression, without said analyses requiring a knowledge of sequence data of the biological materials to be studied or of the specific genes. According to the invention, the nucleic acids of the cDNA population are fractionated by means of the, for example two-dimensional, gel system provided according to the invention and depicted in this way in the form of complex spot patterns. Qualitative and quantitative evaluation thus enables various spot patterns and therefore various DNA samples or mRNA populations to be compared with one another and identified, without the need for previous knowledge of sequence information. In addition, it is also possible according to the invention to isolate individual spots out of the separating system and subject them to sequencing.

In the context of the present invention, a biological material means a material which contains genetic information and which can reproduce itself or can be reproduced in a biological system. Examples of such biological materials are organisms, viruses, cells, yeast, bacteria, cell systems, tissues or the like. In a particularly preferred embodiment, the present invention provides for biological material as starting material, whose genetic information is unknown, i.e. no sequence information of the genetic material thereof is available or is available only in the form of incomplete fragments. In a particularly preferred embodiment, biological material is considered to be, for example, a useful animal such as goats, sheep, horses, dogs, pigs or the like. The invention of course also encompasses plants, in particular crop plants such as rye, barley, tritical, corn, wheat, oats, millet, sugarcane, mangold, sugarbeet, rice, and the like.

In the context of the present invention, a spot means a spatially focused, local accumulation of cDNA molecules obtained after fractionation of the cDNA population, which cDNA molecules have behaved in the same way in the multi-dimensional field and accordingly have essentially the same position within the separating system.

In a further preferred embodiment, the invention provides for the RNA isolated from the biological material in the first step, i.e. total RNA or at least one mRNA population, to be labeled during or after reverse transcription to give a population of double-stranded cDNA, it being possible for the label to be a radiolabel or a fluorescent label, for example. The invention also provides for a labeling by means of, in particular multiple, especially two or three, intercalating dyes, for example Cyber Green, SYBR-Green or SYBR-Gold.

In a particularly preferred embodiment, the invention provides for the multi dimensional fractionation of at least one cDNA population to be two-dimensional or a three-dimensional fractionation. A preferred embodiment of the present invention may provide for the at least one cDNA population to be fractionated in one dimension according to its molecular weight, and, in a further preferred embodiment, high-resolution DNA gel electrophoresis, for example with the use of polyacrylamide gel, is employed for this purpose.

A further embodiment of the invention provides for the at least one cDNA population to be fractionated in one dimension according to its GC content, and, in a further preferred embodiment, fractionation by GC content takes place in a denaturing gradient gel electrophoresis.

In a particularly preferred embodiment of the present invention, fractionation by GC content may also be carried out by means of a temperature gradient gel electrophoresis.

A particularly preferred embodiment provides for a two-dimensional high-resolution fractionation of cDNA molecules from complex populations or mixtures, which fractionation is carried out in one dimension by molecular weight and in a second dimension by GC content.

The quantitative and/or qualitative evaluation of the spot patterns obtained after multidimensional fractionation in the separating system may be carried out by means of commercial scanners, for example.

A preferred embodiment of the invention provides for individual or a plurality of the spots obtained to be isolated from the separating system by means of common methods and subsequently to be subjected to sequence analysis by means of common methods.

In a preferred embodiment, the invention of course also provides for a plurality of different total RNAs or mRNA populations, for example two different mRNA populations, by means of the methods of the invention, to be reverse-transcribed simultaneously to give a plurality of different cDNA populations, to be amplified, where appropriate, and subsequently to be subjected together to a multidimensional fractionation in a separating system. In this embodiment, it is advantageous to label the various cDNA populations obtained differently.

The present method is particularly suitable for use in analysis, diagnostics, in the field of drug discovery and in basic research. In addition, the present method can advantageously be employed in the area of plant cultivation or veterinary medicine. It is essential in all of the areas mentioned to obtain data and findings on the gene expression of biological materials, i.e. findings on, for example, the location-, or time-, or development-specific activity of genes of said biological material, in particular of the organism, without the knowledge of sequence information on the target organisms and/or target genes. The method of the invention enables expressed genes of a biological material to be analyzed and identified rapidly and efficiently, without previous knowledge of sequence information on said biological material or using said knowledge, and it is perfectly possible, depending on the resolution of the separating system employed, for example a two-dimensional gel, to achieve patterns of up to a thousand different spots per separating system, which make an unambiguous characterization analysis of gene expression possible. The present method also guarantees the availability of sequence information by way of subsequent sequence analysis.

The method of the invention may be used advantageously for identifying potential virulence factors in microorganisms, for example yeasts, in particular Candida, preferably Candida albicans, preferably within the framework of gene expression studies.

Further advantageous embodiments arise from the dependent claims.

The invention is further illustrated on the basis of the following exemplary embodiments.

The corresponding figure depicts:

The result of a two-dimensional DNA gel electrophoresis, namely a representative spot pattern of Candida albicans cDNAs.

### EXAMPLE 1

Important properties of the quality of pork are defined essentially by the composition and structural characteristics of the muscle fibers in the muscle tissue. The establishment of favorable properties is determined very much by genetic, predisposing factors. Such factors are identified by taking both muscle tissue from high-quality pork and a tissue sample of low-quality pork fibers. Total RNA is isolated from each of the two samples, and the mRNA present therein is then transcribed into double-stranded cDNA (ds-cDNA) by means of reverse transcription in the presence of differently labeled primers and nucleotides. This cDNA is cut by sequence-specific restriction endonucleases in order to produce ds-DNA fragments of a defined length. The differently labeled ds-cDNA pools are combined and fractionated simultaneously with high resolution by means of multidimensional gel electro-phoresis. This involves fractionating in the first dimension the different fragments by their particular molecular weight in a polyacrylamide gel. The fragments are then further separated by their particular GC content in the second dimension at a right angle to the fractionation in the first dimension. Here, the DNA fragments migrate along a continuous gradient of varying hybridization stringency, with the formamide and urea concentrations steadily increasing. After two-dimensional fractionation of the labeled DNA fragments, the complex spot patterns are visualized by means of fluorescence scanners and then analyzed quantitatively and qualitatively. Spots that can be detected exclusively in samples of high-quality meat and therefore constitute potential markers are excised from the gel and identified by means of DNA sequencing.

### EXAMPLE 2

Human pathogenic yeasts have developed specific virulence mechanisms in order to be able to colonize tissues in the respective host and therefore be capable of causing infections. Thus, for example, Candida albicans causes systemic infections associated with a high mortality rate. In order to identify potential virulence factors in C. albicans, gene expression studies are carried out on clinical isolates of C. albicans under various conditions. For this purpose, total RNA or mRNA is isolated from C. albicans cells, and the corresponding cDNA is then prepared by means of reverse transcription in a manner known per se. The cDNA prepared in this way is digested by means of restriction endonucleases, and defined adapters are subsequently ligated to the thus fragmented cDNA. This adapter-ligated cDNA can then be amplified by means of PCR and fractionated by means of two-dimensional DNA gel electrophoresis. The spot patterns obtained in this way may then be evaluated qualitatively and quantitatively, and individual spots can be punched out, amplified by means of PCR and then sequenced. The individual experimental steps are listed in detail below:

I. Cultivation of Candida albicans Cells and Preparation of Cell Beads

To isolate total RNA, a preculture is established by inoculating 11 ml of YPD medium with Candida albicans cells (clinical isolate SC5314) and agitating the culture at 30° C. overnight. Subsequently, 50 ml of YPD medium are inoculated with overnight culture to give an OD600 of 0.15-0.20. This culture is grown to an OD of 0.8-1.0 on a shaker (180 rpm) at 30° C. The cell beads are prepared by centrifuging the cell suspension at 3000×g for 4 min, resuspending the pellet in the remaining 1.0-1.5 ml of medium and pipetting the suspension dropwise into a 50 ml tube containing liquid nitrogen. The samples frozen in this manner may be stored at −80° C. until isolation.

### II. Isolation of Total RNA

An RNeasy Midi Kit (Qiagen) may be used for isolating total RNA from C. albicans and also from eukaryotic cells. However, the cells are not disrupted with the aid of glass beads but by using a ball mill from Retsch, for example.

The cell beads obtained are crushed by a tungsten carbide sphere in Teflon containers previously cooled with liquid nitrogen, to give a fine powder (shaking frequency: 30 s⁻¹, time: 2 min) which is then transferred to the lysis buffer (buffer RLT+0.01% (v/v) mercaptoethanol) of the kit. The sample is vortexed, i.e. agitated vigorously, for 1 min for homogenization. The solid components of the lysate are then first centrifuged at 3500×g for 5 min, and the supernatant is admixed with one volume of 70% ethanol and mixed well. All of this solution is then applied in 4 ml aliquots to the RNeasy Midi column and centrifuged at 3500×g for 5 min each. The RNA bound to the membrane is then washed according to the manufacturer's instructions, first with 4 ml of RW1 buffer, then twice with in each case 2.5 ml of RPE buffer. The RNA is eluted by pipetting 250 μl of RNase-free water onto the membrane, incubating the column at room temperature for 1 min and then centrifuging at 3500×g for 3 min. These steps are repeated once, resulting in a final volume of 500 μl.

### III. Precipitation of Total RNA

Total RNA is precipitated by transferring in each case 250 μl of eluate to two 1.5 ml reaction vessels and precipitating at −20° C. overnight after adding 25 μl of 3 mol/l sodium acetate, pH 5.3, and 625 μl of 100% ethanol (−20° C.). The precipitated nucleic acids are pelleted by centrifuging at 4° C., 13000×g for 30 minutes and then washed twice with 1 ml of 70% EtOH-DEPC (−20° C.) to remove any salt residues still present.

After drying the total RNA precipitate at 37° C., the RNA is taken up in 10-50 μl of DEPC-H₂O, and concentration and purity are determined by means of UV spectrometry.

IV. Preparation of ds-cDNA from polyA+ or Total RNA

Full length ds-cDNA is synthesized from 0.5-1 μg of polyA+ or total RNA according to a modified protocol from the SMART cDNA Library Construction Kit (BD Bioscience).

Primers used (in each case 10 mol/l):

### SMART IV Oligonucleotide:

(SEQ ID No. 1)

5′-AAGCAGTGGTATCAACGCAGAGTGGCCATTACGGCCGGG-3′
ODD_T_all:

(SEQ ID No. 2)

5′-GCGAGTCGACCGTTTTTTTTTTTTT-3′
ODD-TA:

(SEQ ID No. 3)

5′-GCGAGTCGACCGTTTTTTTTTTTTTA-3′
ODD-TG:

(SEQ ID No. 4)

5′-GCGAGTCGACCGTTTTTTTTTTTTTG-3′
ODD-TC:

(SEQ ID No. 5)

5′-GCGAGTCGACCGTTTTTTTTTTTTTC-3′
A2-Sau3a:

(SEQ ID No. 6)

5′-AAGCAGTGGTATCAACGCAGAGT-3′

To synthesize the first strand of the cDNA, 1-3 μl of RNA [0.5-1.0 μg of polyA+ or total RNA] is admixed with in each case 1 μl of 10 μmol SMART IV oligonucleotide and ODD_T_all primers (alternatively: a 10 μmol/1 mix of ODD-TA, ODD-TG and ODD-TC) in an RNase-free reaction vessel, with DEPC H₂O being added to give a total volume of 5 μl.

To denature the RNA secondary structure, the reaction mixture is incubated first at 72° C. for 2 min and then on ice for 2 min. This is followed by adding 2 μl of 5×first strand buffer (250 mmol/l Tris, pH 8.3; 30 mmol/l Mg/Cl₂; 375 mmol/l KCl), 1 μl of 20 mmol/l DTT, 1 μl of 10 mmol/l dNTP mix and 1 μl of PowerScript reverse transcriptase and mixing the components by carefully pipetting up and down. The reverse transcriptase reaction is carried out at 42° C. for 1 h and is stopped by transfer onto ice. This first strand cDNA may then either be used directly in the second strand synthesis or stored up to three months at −20° C.

The second strand is synthesized using the BD Advantage™ 2 Polymerase Mixes in Long Distance (LD) PCR. This mixture is BD TITANIUM™ Taq DNA polymerase in combination with BD TaqStart™ antibodies and a small amount of a proofreading polymerase.

In a total volume of 100 μl, 2 μl of first strand cDNA, 80 μl of molecular-biological H 20, 10 μl of 10×Advantage 2 PCR buffer (400 mmol/l Tricine-KOH, pH 8.7; 150 mmol/l KOAc; 35 mmol/l Mg(OAc)₂; 37.5 μg/ml BSA; 0.05% Tween 20; 0.05% Nonidet-P40), 2 μl of 50×dNTP mix (10 mmol/l each), 2 μl of A2-Sau3A primer, 2 μl of ODD_T_all primer (alternatively: ODD-TA/TC/TG primer mix) and 2 μl of 50×Advantage™ 2 polymerase mix are combined, briefly vortexed, i.e. agitated vigorously, followed by two cycles of “Hot Start PCR”.

PCR program:

95° C.-0:20 min

95° C.-0:05 min

68° C.-6:00 min

95° C.-0:05 min

68° C.-6:00 min

V. Purification of ds-cDNA

ds-cDNAs are purified by means of column purification (e.g. QIAGEN QIAquick PCR purification kits. Elution is carried out by adding 90 μl of EB buffer (10 mmol/l Tris-Cl, pH 8.5).

VI. Restriction Digestion of ds-cDNA by Restriction Endonuclease (e.g. RsaI)

In a 100 μl digestion mixture, 90 μl of the eluate are admixed with 2 μl of 10× buffer 1 (100 mmol/l Bis-Tris propane HCl, pH 7.0; 100 mmol/l MgCl 2; 10 mmol/l dithioreitol) and 12 μl of 10 mg/ml BSA and, after addition of 30 U of RsaI, incubated at 37° C. for 2 h. After further addition of 15 U of RsaI and incubation for 1 h, the cut ds-cDNA can be precipitated.

VII. Precipitation of Fragmented ds-cDNA

The cDNA is precipitated by adding 0.1 volume of 3 mol/l sodium acetate, pH 5.3, and 2.5 volumes of 100% EtOH directly to the restriction digest and incubating at −20° C. for 2 h. The pellet is obtained by centrifugation (13 000×g) in a refrigerated centrifuge at 4° C. for 25 minutes. After washing in 70% ethanol [−20° C.], the pellet may be taken up in 10 μl of molecular-biological water. 5 μl thereof are used for adaptor ligation and the remainder is frozen at −20° C.

### VIII. Adapter Synthesis

### Oligonucleotides Used:

Long Oligo RsaI [100 μmol/l]:

(SEQ ID No. 7)

GCGTGAAGACGACAGAAAGGGCGTGGTGCGGAGGGCGGT
Short Oligo RsaI [100 μmol/l]:

(SEQ ID No. 8)

ACCGCCCTCCGC

A 20 μmol/l adaptor stock (ODD_Adapter_RSaI) is prepared by adding in each case 20 μl of Long Oligo RsaI and Short Oligo RsaI primers, 50 mmol/l Tris HCl, pH 7.5; mmol/l MgCl 2; 10 mmol/l dithiothreitol; 1 mmol/l ATP; 25 μg/ml BSA in a total volume of 100 μl. Upon 3 min of denaturation at 95° C. for 32 min and subsequent slow cooling (approx. 8 h) at RT, the complementary nucleotide sequences hybridize to give double-stranded adaptors which may be used for ligation of RsaI-digested ds-cDNA.

### IX. Adapter Ligation

5 μl of the ds-cDNA precipitated after RsaI digestion are used for adapter ligation.

The ligation is carried out by mixing 1 μl of the μmol/l adapter stock solution [ODD_Adapter_RsaI], 1 μl of 10×T4 ligase buffer (500 mmol/l Tris HCl, pH 7.5; 100 mmol/l MgCl₂; 100 mmol/l di thiothreitol; 10 mmol/l ATP; 250 μg/ml BSA), 5 μl ds-cDNA and 200 U of T4 DNA ligase in a total volume of 10 μl, followed by incubation at 16° C. over night.

Ligase activity is stopped by way of heat inactivation (10 min at 65° C.) on the next day.

The 10 μl ligation mixture is then admixed with 90 μl of water to give 100 μl and purified by means of columns (QIAGEN PCR purification kit protocol).

X. ADAPTER-PCR (Amplification of the 3′ ends of RsaI-Digested ds-cDNA)

“Suppression PCR” using the primers ODD_T_all and Primer AP1 is used especially for amplifying the 3′ ends of cDNA.

The adapter-ligated DNA is amplified by the following PCR:

10 μl off the eluate of the adapter-ligated ds-cDNA are mixed with 2.5 μl of 10×PCR buffer (200 mmol/l Tris HCl, pH 8.4; 500 mmol/l KCl), 0.6 μl of 50 mmol/l MgCl₂, 0.05 μl of 5 mmol/l dNT{s, 0.25 μl of 10 μmol/l AP1 primer

| | |
|---|---|
| (5′-TGTAGCGTGAAGACGACAGAA-3′), | (SEQ ID No. 9) |

0.25 μl ODD_T_all primer

| | |
|---|---|
| (5′-GCGAGTCGACCGTTTTTTTTTTTTT-3′) | (SEQ ID No. 2) |

10.65 μl of molecular-biological water and 2 U of Taq polymerase are subsequently amplified using the following PCR program:

PCR program:

### 1. 95° C.-0:05 min

### 2. 65° C.-0:30 min

### 3. 72° C.-1:00 min

Steps 2 and 3 are repeated 19 times (total number of cycles: 20).

The cDNA amplified in this way may then be amplified specifically in a second PCR (adapter PCR II) using “anchored primers”

### Primer 1:

| | |
|---|---|
| AGGGCGTGGTGCGGAGGGCGGTCCNN | (SEQ ID No, 10) |

where N may represent the nucleotides A, G, C or T, resulting in 16 possible primer 1 variants; and

### Primer 2:

| | |
|---|---|
| 5′-GCGAGTCGACCGTTTTTTTTTTTTTVN-3′, | (SEQ ID No. 11) |

where V may represent the nucleotides A, G or C and N may represent the nucleotides A, G, C or T, thus resulting in 12 possible primer 2 variants).

### XI Adapter PCR II

The following reaction mixture is chosen for the specific amplification of subpopulations of amplified cDNA fragments from adapter PCRI:

The amplified cDNA fragments from adapter PCR I are diluted 1:30 with water. 10 μl of this dilution are mixed with 10 μl of 10×PCR buffer (200 mmol/l Tris HCl, pH 8.4; 500 mmol/l KCl), 3 μl of 50 mmol/l MgCl₂, 4 μl of 5 mmol/l of dNTPs, 2.5 μl of 10 μmol/l oligonucleotide primer 1

| | |
|---|---|
| (5′-GGGCGTGGTGCGGAGGGCGGTCCNN-3′), | (SEQ ID No. 10) |

2.5 μl of 10 μmol/l of Primer 2

| | |
|---|---|
| (5′-GCGAGTCGACCGTTTTTTTTTTTTVN-3′) | (SEQ ID No. 11) |

65.5 μl of molecular-biological water and 20 U of Taq polymerase, and subsequently amplified using the following PCR program:

PCR program:

### 1. 95° C.-0:05 min

### 2. 65° C.-0:30 min

### 3. 72° C.-1:00 min

Steps 2 and 3 are repeated 20 times (total number of cycles: 21).

XII. Two-Dimensional cDNA Fractionation

The amplified cDNA fragments from adapter PCR II are then fractionated with high resolution by means of two-dimensional gel electrophoresis. For this purpose, the samples are first applied, in the first dimension, to a non-denatured polyacrylamide gel. Composition of the gel for the first dimension (fractionation by molecular weight): 8% acrylamide (acrylamide:bisacrylamide ratio of 37.5:1) in 1×TAE (40 mmol/l Tris; 20 mmol/l glacial acetic acid; 1 mmol/l EDTA, pH 8.0). The samples are electrophorized at a constant voltage of 200 volt for 2-4 hours. Subsequently, the lane of the first dimension is excised and put on top of a polyacrylamide gel containing a concentration gradient of urea and formamide for fractionation in the second dimension. The polyacrylamide gel of the second dimension may have either a constant concentration or likewise a concentration gradient of acrylamide. Composition of the gel for the second dimension: 8% acrylamide (acrylamide:bisacrylamide ratio of 37.5:1) in 1×TAE (40 mmol/l Tris; 20 mmol/l glacial acetic acid; 1 mmol/l EDTA, pH 8.0); continuous gradient of 0.7 mol/l urea, 4% formamide at the top, to 2.8 mol/l urea, 16% formamide at the bottom. The fragments are electrophorized in this second dimension at a constant temperature of 60° C. and a constant voltage of 100 volt for 15 hours.

### XIII. Detection of DNA Fragments

The 2D gel is stained by means of SYBR-Gold for visualizing the DNA spots. If fluorescently labeled primers 1 and 2 are used in adapter PCR II, the 2D gel is not stained by means of SYBR-Gold. The DNA spots are detected by means of commercial fluorescence documentation systems.

The figure depicts a representative spot pattern for C. albicans cDNAs after two-dimensional fractionation and staining by means of SYBR-Golg.

1st dimension: 8% TAE polyacrylamide gel (fractionation by molecular weight)
2nd dimension: denaturing gradient gel electrophoresis, 8% acrylamide, continuous denaturing gradient from 4% formamide+0.7 mol/l urea to 16% formamide+2.8 mol/l urea (fractionation by GC content).

1. A method of quantitative or qualitative analysis of gene expression of a biological material, comprising:
isolating and precipitating total RNA from biological material;
preparing double-stranded cDNA from the total RNA;
restriction digesting the cDNA by restriction endonuclease to produce fragmented cDNA;
precipitating the fragmented cDNA;
ligating of cDNA fragments with adapters;
carrying out an amplification reaction for amplifying the 3′ ends of the ligated fragments and, where appropriate, specifically amplifying subpopulations thereof;
carrying out a two-dimensional fractionation of amplified cDNA, with the cDNA population being fractionated according to its molecular weight in the first dimension and according to its GC content in the second dimension; and
detecting and at least one of qualitative and quantitatively evaluating multidimensionally fractionated spots obtained of the at least one cDNA population.

2. The method according to claim 1, wherein the fractionation by molecular weight is carried out by a DNA gel electrophoresis system.

3. The method according to claim 1, wherein the fractionation by GC content is carried out in a denaturing gradient gel electrophoresis.

4. The method according to claim 1, wherein in a further step sequencing of DNA molecules from at least one of the cDNA spots obtained in the separating system and isolated therefrom is carried out.

5. The method according to claim 1, wherein the double-stranded cDNA population is labeled with intercalating dyes or is radiolabeled or fluorescently labeled.

6. A method of quantitative or qualitative analysis of gene expression of a biological material, comprising:
isolating in a first step a) at least two different total RNAs or at least two different mRNA populations from biological material,
obtaining in a second step b) at least two different populations of double-stranded cDNA from the at least two different RNAs, wherein the at least two different populations of double-stranded cDNAs are labeled in each case differently during this step,
carrying out in a third step c) a joint multidimensional distribution of the at least two different cDNA populations in a separating system, and
carrying out in a fourth step d) at least one of a qualitative and a quantitative evaluation of the multidimensionally fractionated spots obtained of the at least two cDNA populations.

7. The method according to claim 2, wherein the fractionation by GC content is carried out in a denaturing gradient gel electrophoresis.

8. The method according to claim 6, wherein said at least two different populations of double-stranded cDNA are obtained by reverse transcription.

9. The method according to claim 6, wherein said at least two different populations of double-stranded cDNA are at least two different total RNAs.

10. The method according to claim 6, wherein said at least two different populations of double-stranded cDNA are at least two different mRNA populations.


SEQUENCE LISTING (11 sequences)

SEQ ID NO: 1 - DNA, 39 bp, Artificial
  Candida albicans
aagcagtggtatcaacgcagagtggccattacggccggg

SEQ ID NO: 2 - DNA, 25 bp, Artificial
  Candida albicans
gcgagtcgaccgttttttttttttt

SEQ ID NO: 3 - DNA, 26 bp, Artificial
  Candida albicans
gcgagtcgaccgttttttttttttta

SEQ ID NO: 4 - DNA, 26 bp, Artificial
  Candida albicans
gcgagtcgaccgtttttttttttttg

SEQ ID NO: 5 - DNA, 26 bp, Artificial
  Candida albicans
gcgagtcgaccgtttttttttttttc

SEQ ID NO: 6 - DNA, 23 bp, Artificial
  Candida albicans
aagcagtggtatcaacgcagagt

SEQ ID NO: 7 - DNA, 39 bp, Artificial
  Candida albicans
gcgtgaagacgacagaaagggcgtggtgcggagggcggt

SEQ ID NO: 8 - DNA, 12 bp, Artificial
  Candida albicans
accgccctccgc

SEQ ID NO: 9 - DNA, 21 bp, Artificial
  Candida albicans
tgtagcgtgaagacgacagaa

SEQ ID NO: 10 - DNA, 26 bp, Artificial
  Candida albicans
agggcgtggtgcggagggcggtccnn

SEQ ID NO: 11 - DNA, 27 bp, Artificial
  Candida albicans
gcgagtcgaccgtttttttttttttvn